(19) United States
(12) Patent Application Publication
(10) Pub. No.: US 2023/0324099 A1
(43) Pub. Date: Oct. 12, 2023

(54) REFRIGERATOR

(71) Applicant: SAMSUNG ELECTRONICS CO., LTD., Suwon-si (KR)

(72) Inventors: Youngmin YOU, Suwon-so (KR); IIsung Bae, Suwon-si (KR); Jinyoung Song, Suwon-si (KR); Byungkwan Yang, Suwon-si (KR); Sangjune Lee, Suwon-si (KR)

(73) Assignee: SAMSUNG ELECTRONICS CO., LTD., Suwon-si (KR)

(21) Appl. No.: 18/209,984

(22) Filed: Jun. 14, 2023

Related U.S. Application Data
(63) Continuation of application No. PCT/KR2021/016246, filed on Nov. 9, 2021.

(30) Foreign Application Priority Data
Jan. 6, 2021 (KR) 10-2021-0001605

(51) Int. Cl. F25D 17/04

(52) U.S. Cl. CPC F25D 17/042

(57) ABSTRACT

A refrigerator includes: an inner case defining a storage compartment; a cold air outlet provided on one side of the storage compartment and configured to discharge cold air; and a purifier assembly provided at an upper surface of the storage compartment and configured to purify a portion of the cold air discharged from the cold air outlet, wherein the purifier assembly includes: an air purifier configured to deodorize or sterilize the cold air; and a housing including the air purifier and an inlet facing the cold air outlet, wherein the inlet is configured to receive the cold air discharged from the cold air outlet, the purifier assembly penetrates the inner case, and a portion of the housing is provided inside the storage compartment and a remaining portion of the housing is provided outside the storage compartment.

### CROSS-REFERENCE TO RELATED APPLICATIONS

This application is a by-pass continuation application of International Application No. PCT/KR2021/016246, filed on Nov. 9, 2021, which is based on and claims priority to Korean Patent Application No. 10-2021-0001605, filed on Jan. 6, 2021, in the Korean Intellectual Property Office, the disclosures of which are incorporated by reference herein their entireties.

### BACKGROUND

### 1. Field

The disclosure relates to a refrigerator, and more particularly, to a refrigerator having an air purifier.

### 2. Description of Related Art

In general, a refrigerator, which is an apparatus for keeping food fresh, is a home appliance provided to supply cold air to food by including a storage compartment for storing food and a cold air supply device for supplying cold air to the storage compartment.

Because various types of food are stored in the storage compartment of the refrigerator, odors or bacteria generated from the foods may inevitably occur inside the storage compartment. Therefore, an air purifier for removing odors or bacteria generated in the storage compartment is required for the refrigerator.

### SUMMARY

Provided is a refrigerator having a structure capable of minimizing a portion of a purifier assembly exposed through a storage compartment.

In addition, provided is a refrigerator in which a type of air purifier may be easily changed.

According to an aspect of the disclosure, a refrigerator includes: an inner case defining a storage compartment; a cold air outlet provided on one side of the storage compartment and configured to discharge cold air; and a purifier assembly provided at an upper surface of the storage compartment and configured to purify a portion of the cold air discharged from the cold air outlet, wherein the purifier assembly includes: an air purifier configured to deodorize or sterilize the cold air; and a housing including the air purifier and an inlet facing the cold air outlet, wherein the inlet is configured to receive the cold air discharged from the cold air outlet, the purifier assembly penetrates the inner case, and a portion of the housing is provided inside the storage compartment and a remaining portion of the housing is provided outside the storage compartment.

The purifier assembly may further include a first flow path and a second flow path in which the cold air received by the inlet flows and which are partitioned from each other, and the air purifier is provided on the first flow path and is further configured to deodorize or sterilize the cold air flowing through the first flow path.

The air purifier may further include at least one of (i) a photocatalytic device including a ultraviolet (UV) light source and a photocatalytic filter configured to react with light from the UV light source, or (ii) an ion sterilization device including a discharge electrode and a printed circuit board configured to control the discharge electrode.

The air purifier may include the photocatalytic device, and the purifier assembly may further include a supporter provided on the first flow path and configured to support the UV light source to be disposed above the photocatalytic filter.

The purifier assembly may further include an electronic component room partitioned from the first flow path and the second flow path, the air purifier may include the ion sterilization device, the discharge electrode may be provided on the first flow path, and the printed circuit board may be provided in the electronic component room.

The housing may further include a mounting portion on which the photocatalytic filter or the discharge electrode is mounted, and the purifier assembly may further include an outlet provided on a bottom surface of the mounting portion and through which the cold air in the first flow path is discharged to the storage compartment.

The inner case may include an insertion hole, and the housing may penetrate the insertion hole.

The refrigerator may further include a fixing cover provided outside the inner case and to which the housing penetrating the insertion hole is fixed.

The housing may further include a coupling hook provided on an upper surface of the housing and configured to be coupled to the fixing cover.

The refrigerator may further include: a duct including a circulation flow path configured to circulate the cold air in the storage compartment and a fan provided on the circulation flow path, the cold air outlet is provided at an upper end of one surface of the duct forming a rear surface of the storage compartment, and the inlet may be configured to receive the cold air discharged from the fan.

The inlet may be provided inside the storage compartment.

The inlet may be adjacent to the cold air outlet.

A flow rate of the cold air discharged from the second flow path to the storage compartment may be greater than a flow rate of the cold air discharged from the first flow path to the storage compartment.

A direction in which the cold air is discharged from the first flow path to the storage compartment may be different from a direction in which the cold air is discharged from the second flow path to the storage compartment.

The refrigerator may further include a deodorization filter provided on a rear surface of the storage compartment.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a perspective view of a refrigerator according to an embodiment of the present disclosure;

FIG. 2 is a front view illustrating a state in which a purifier assembly is separated from a first storage compartment of the refrigerator illustrated in FIG. 1;

FIG. 3 is a front view illustrating a state in which the purifier assembly is mounted in the first storage compartment illustrated in FIG. 2;

FIG. 4 is a cross-sectional view taken by cutting an indicator line A-A′ shown in FIG. 3;

FIG. 5 is an enlarged view of part B shown in FIG. 4;

FIGS. 6 to 9 are views illustrating the purifier assembly illustrated in FIG. 3 at various directions;

FIG. 10 is a view illustrating an inner upper surface of an inner case in the state in which a purifier assembly is separated in the refrigerator illustrated in FIG. 3;

FIG. 11 is a bottom perspective view of a fixing cover;

FIG. 12 is an exploded perspective view illustrating a state in which a photocatalytic device is mounted in the purifier assembly illustrated in FIG. 6;

FIG. 13 is an exploded perspective view showing a state in which an ion sterilizer is mounted in the purifier assembly illustrated in FIG. 6;

FIG. 14 is an enlarged view of part C shown in FIG. 12;

FIG. 15 is a top view of the C part;

FIG. 16 is a view illustrating a state in which a deodorization filter is disposed in the first storage compartment illustrated in FIG. 3; and

FIG. 17 is a flowchart illustrating an operation sequence of the purifier assembly illustrated in FIG. 3.

### DETAILED DESCRIPTION

The embodiments described in the present specification and the configurations shown in the drawings are only examples of preferred embodiments of the present disclosure, and various modifications may be made at the time of filing of the present disclosure to replace the embodiments and drawings of the present specification.

Like reference numbers or signs in the various drawings of the application represent parts or components that perform substantially the same functions.

The terms used herein are for the purpose of describing the embodiments and are not intended to restrict and/or to limit the present disclosure. For example, the singular expressions herein may include plural expressions, unless the context clearly dictates otherwise. Also, the terms “comprises” and “has” are intended to indicate that there are features, numbers, steps, operations, elements, parts, or combinations thereof described in the specification, and do not exclude the presence or addition of one or more other features, numbers, steps, operations, elements, parts, or combinations thereof.

It will be understood that, although the terms first, second, etc. may be used herein to describe various components, these components should not be limited by these terms, and these terms are only used to distinguish one component from another. For example, without departing from the scope of the present disclosure, the first component may be referred to as a second component, and similarly, the second component may also be referred to as a first component. The term “and/or” includes any combination of a plurality of related items or any one of a plurality of related items.

In this specification, the terms “front end,” “rear end,” “upper portion,” “lower portion,” “upper end” and “lower end” used in the following description are defined with reference to the drawings, and the shape and position of each component are not limited by these terms. Based on the coordinate axes shown in the drawings, an X-axis direction may be referred to as a front-back direction, a Y-axis direction may be referred to as a left-right direction, and a Z-axis direction may be referred to as an up-down direction.

Hereinafter, embodiments of the present disclosure will be described in detail with reference to the accompanying drawings.

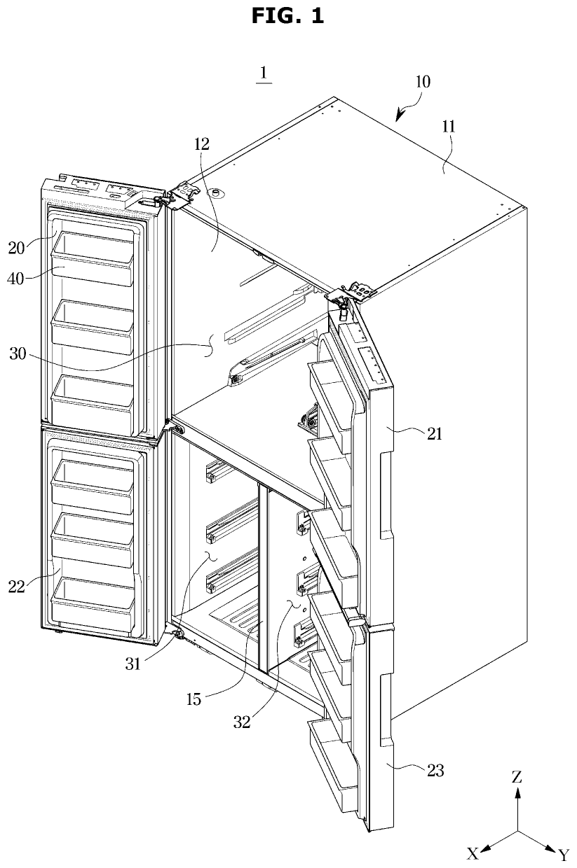

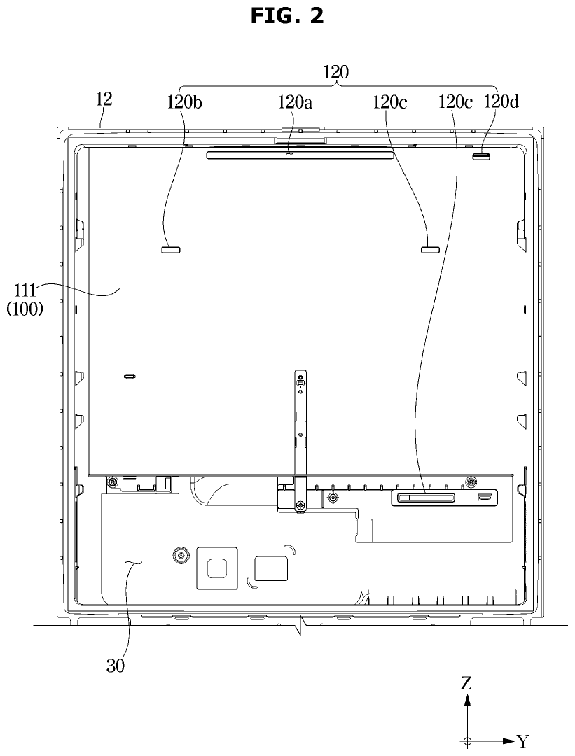

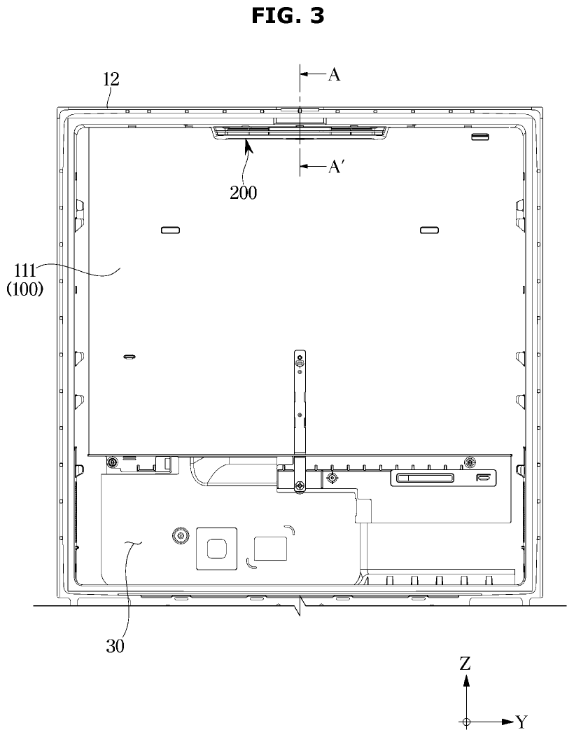

FIG. 1 is a perspective view of a refrigerator according to an embodiment of the present disclosure. FIG. 2 is a front view illustrating a state in which a purifier assembly is separated from a first storage compartment of the refrigerator illustrated in FIG. 1. FIG. 3 is a front view illustrating a state in which the purifier assembly is mounted in the first storage compartment illustrated in FIG. 2.

Referring to FIGS. 1 to 3, a refrigerator 1 may include a main body 10 and storage compartments 30, 31, and 32 provided inside the main body 10 and having an open front so that objects may be taken in and out.

The main body 10 may include an inner case 12 and an outer case 11. The outer case 11 may be provided to form an exterior of the refrigerator 1. The outer case 11 may be coupled to the outside of the inner case 12. The outer case 11 may be formed of a metal material having excellent durability and aesthetics. However, the material of the outer case 11 is not limited thereto.

The inner case 12 may be disposed inside the outer case 11. Storage compartments may be formed inside the inner case 12. The inner case 12 may be made of a plastic material to be integrally injection molded.

A heat insulating material is foamed between the inner case 12 and the outer case 11 to secure insulation of the refrigerator 1.

The refrigerator 1 may include a plurality of the storage compartments 30, 31, and 32. The storage compartments may include the first storage compartment 30, the second storage compartment 31, and the third storage compartment 32. Especially, the second storage compartment 31 and the third storage compartment 32 may be partitioned by a partition wall 15.

The first storage compartment 30 may be provided at an upper portion of the refrigerator 1, and the second storage compartment 31 and the third storage compartment 32 may be divided and provided at a lower portion of the refrigerator 1. However, a shape and number of the storage compartments 30, 31, and 32 are not limited thereto.

The refrigerator 1 may include a refrigerating compartment and a freezing compartment. Depending on a type of refrigerator 1, the first upper storage compartment 30 may be provided as the refrigerating compartment, and the second lower storage compartment 31 and the third lower storage compartment 32 may be provided as the freezing compartments. However, positions of the refrigerating compartment and the freezing compartments are not limited thereto.

The refrigerator 1 illustrated in FIG. 1 may be the refrigerator 1 in which the first storage compartment 30 positioned at the upper portion may be provided as the refrigerating compartment, and the second storage compartment 31 and the third storage compartment 32 positioned at the lower portion may be provided as the freezing compartments. The refrigerating compartment may be kept at approximately 3 degrees Celsius and the freezing compartments may be kept at approximately minus 20 degrees Celsius.

The storage compartments 30, 31, and 32 may be opened and closed by doors 20, 21, 22, and 23. The first storage compartment 30 may be opened and closed by the first door 20 and the second door 21 rotatably coupled to the main body 10. The second storage compartment 31 may be opened and closed by the third door 22 rotatably coupled to the main body 10. The third storage compartment 32 may be opened and closed by the fourth door 23 rotatably coupled to the main body 10. However, the present disclosure not limited thereto, and the doors 20, 21, 22, and 23 may be provided as various forms as long as they may open and close the storage compartments 30, 31, and 32.

A plurality of door guards 40 capable of storing objects and the like may be provided on rear surfaces of the first door 20 to the fourth door 23. The door guard 40 may be provided to accommodate objects and the like having small volume.

The refrigerator 1 may include a cooling system provided to supply cold air to the storage compartments 30, 31, and 32.

The cooling system may include a compressor, a condenser, an expansion valve, an evaporator, and a blowing fan. The cooling system may be disposed in a machine room provided at a lower rear side of the main body 10.

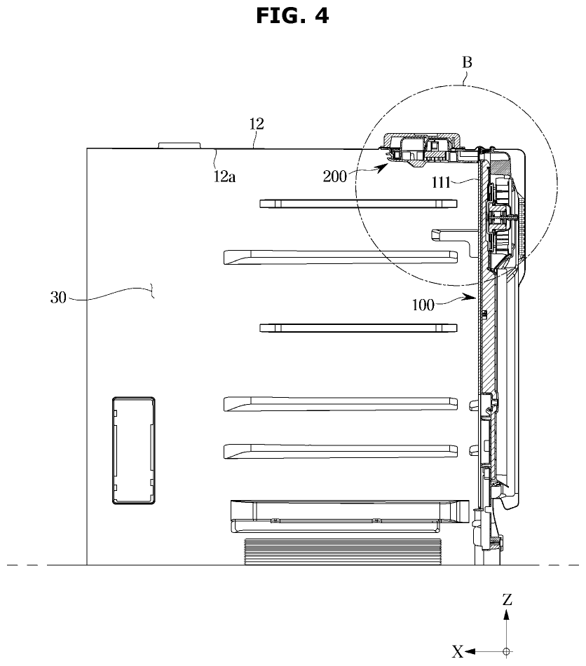

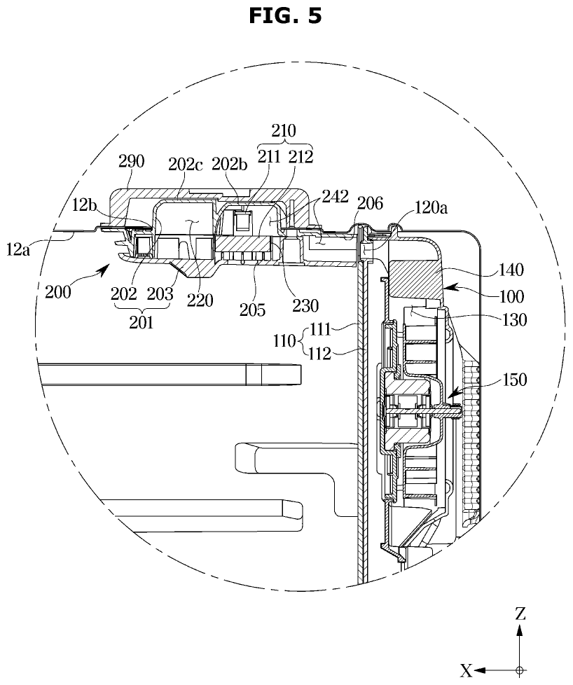

The refrigerator 1 may include a duct 100 (see FIG. 4) forming a circulation flow path 130 (see FIG. 5) provided to flow cold air generated through the cooling system into the storage compartments 30, 31, and 32.

The refrigerator 1 may include a cold air outlet 120 for supplying the cold air delivered through the duct 100 to the storage compartments. The cold air outlet 120 may include a plurality of cold air discharge ports 120a to 120e. The cold air outlet 120 may be provided at a rear surface of the storage compartments 30, 31, and 32.

FIGS. 2 and 3 illustrate the cold air outlet 120 and a purifier assembly 200 in the first storage compartment 30, and the purifier assembly 200 in the first storage compartment 30 will be described in detail below. Hereinafter, the storage compartment refers to the first storage compartment 30. However, the present disclosure is not limited thereto, and separate purifier assemblies may also be provided in the second storage compartment 31 and the third storage compartment 32.

The refrigerator 1 may include the purifier assembly 200 provided to purify a portion of the cold air discharged from the cold air discharge port 120a disposed at an uppermost upper portion among a plurality of the cold air discharge ports of the cold air outlet 120. The purifier assembly 200 may be fixed to an upper surface of the storage compartment 30 to correspond to the upper cold air discharge port 120a. In general, because a greater flow rate of cold air is discharged through the upper cold air discharge port 120a than through the other cold air discharge ports 120b to 120e, as the purifier assembly 200 purifies the cold air discharged from the upper cold air discharge port 120a, purification efficiency may be increased. Hereinafter, the cold air outlet 120 refers to the upper cold air discharge port 120a.

Although not separately shown, a plurality of shelves may be disposed in the inner case 12. The plurality of shelves may be provided to be disposed in front of the duct 100 to accommodate objects.

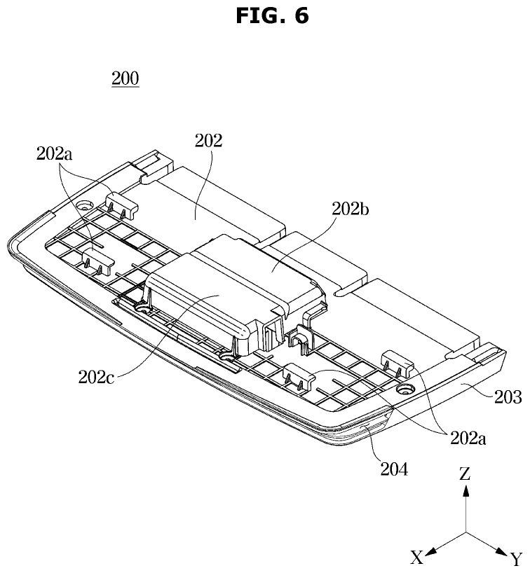

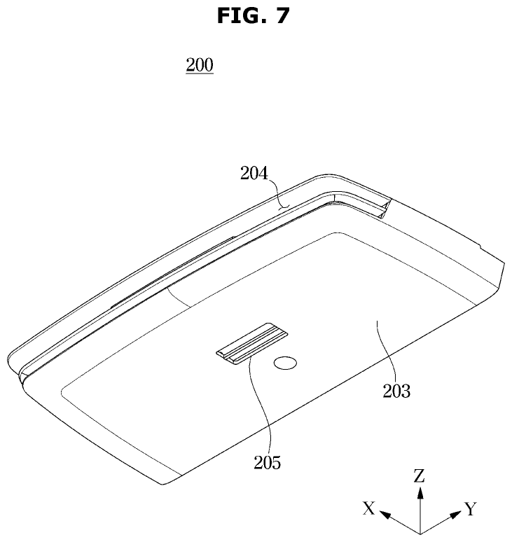

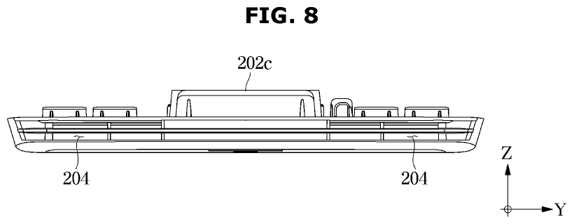

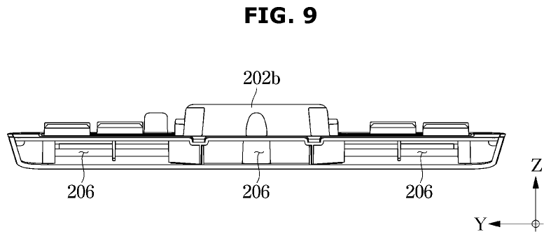

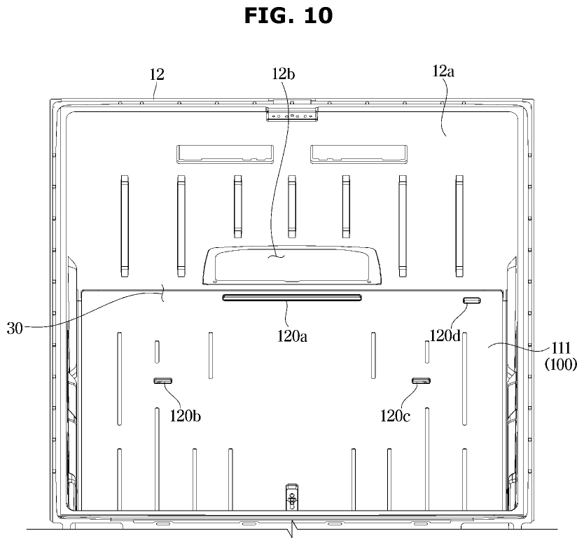

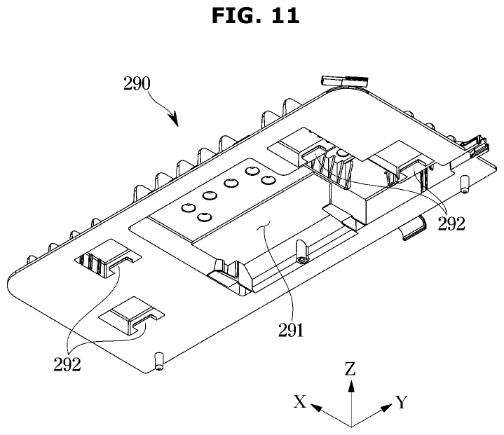

FIG. 4 is a cross-sectional view taken by cutting an indicator line A-A′ shown in FIG. 3. FIG. 5 is an enlarged view of part B shown in FIG. 4. FIGS. 6 to 9 are views illustrating the purifier assembly illustrated in FIG. 3 at various directions. FIG. 10 is a view illustrating an inner upper surface of an inner case in the state in which a purifier assembly is separated in the refrigerator illustrated in FIG. 3. FIG. 11 is a bottom perspective view of a fixing cover.

Hereinafter, a structure of fixing the purifier assembly to the inner case will be described in detail with reference to FIGS. 4 to 11.

The duct 100 may be mounted in the inner case 12 of the refrigerator 1. The duct 100 may be provided in the storage compartment 30. The duct 100 may be mounted in the inner case 12 to form the circulation flow path 130 for supplying cold air to the storage compartment 30. The duct 100 may be mounted on a rear surface of the inner case 12. Accordingly, when a user opens the first door 21 or the second door 22, the duct 100 may be disposed toward the front to face the user. More specifically, a front cover 111 of the duct 100 may be seen in a field of view of the user by being disposed to be exposed to the storage compartment 30. That is, the front cover 111 may form the rear surface of the storage compartment 30.

The duct 100 may be mounted on the rear surface of the inner case 12 to cover most of the rear surface. A surface of the duct 100 may be provided to be flat. However, the surface shape of the duct 100 is not limited thereto.

The duct 100 may include a support plate 112 provided to support the front cover 111. The front cover 111 may be detachably provided on the support plate 112. The support plate 112 may constitute a cover unit 110 together with the front cover 111.

The duct 100 may include a rear cover 140 coupled to the support plate 112 and forming the circulation flow path 130 through which the cold air flows. Specifically, the circulation flow path 130 may be formed between the support plate 112 and the rear cover 140.

The cold air discharge port 120a may communicate with the circulation flow path 130 by penetrating the front cover 111 and the support plate 112. That is, the cold air discharge port 120a may penetrate the cover unit 110. Cold air flowing through the circulation flow path 130 may be discharged through the cold air discharge port 120a. The cold air discharge port 120a may be disposed on the front cover 111. The cold air discharge port 120a may be provided at an upper end of the front cover 111. The cold air discharge port 120a may be provided at an upper end of the cover unit 110.

The duct 100 may include a fan 150. The fan 150 may be mounted in the rear cover 140. The fan 150 may be provided to assist a flow of cold air generated by the cooling system and flowing through the circulation flow path 130.

The refrigerator 1 may include the purifier assembly 200 provided to purify a portion of the cold air discharged from the cold air discharge port 120a.

The purifier assembly 200 may include a housing 201 forming an exterior, and an air purifier 210 accommodated inside the housing 201 to deodorize or sterilize cold air.

The housing 201 may include a first housing 203 forming a lower surface of the purifier assembly 200, and a second housing 202 disposed at an upper portion of the first housing 203 to form an upper surface of the purifier assembly 200. The first housing 203 may be coupled to the second housing 202. However, the present disclosure is not limited thereto, and the first housing 203 and the second housing 202 may be integrally formed.

The purifier assembly 200 may include an inlet 206 corresponding to the cold air discharge port 120a to face the cold air discharge port 120a. The inlet 206 may be formed at a rear end of the housing 201. The inlet 206 may be open toward the rear. The inlet 206 may be formed by the first housing 203 and the second housing 202. Cold air discharged from the cold air discharge port 120a may be introduced into the housing 201 through the inlet 206.

The inlet 206 may be disposed adjacent to the cold air discharge port 120a. The inlet 206 may be in contact with the cold air discharge port 120a or may be spaced apart from each other. Cold air discharged from the cold air discharge port 120a may be directly introduced into the inlet 206. The inlet 206 may cover the cold air discharge port 120a.

The purifier assembly 200 may include first and second outlets 204 and 205 through which cold air discharged from the cold air discharge port 120a and introduced into the inlet 206 is discharged to the storage compartment 30. The first outlet 205 may discharge cold air downward. The second outlet 204 may discharge cold air forward.

The purifier assembly 200 may be fixed to one surface of the inner case 12. The purifier assembly 200 may be fixed to an upper surface 12a of the inner case 12. The upper surface 12a of the inner case 12 may form the upper surface of the storage compartment 30.

Specifically, the inner case 12 may include an insertion hole 12b formed on the upper surface 12a of the inner case 12. The housing 201 may penetrate the insertion hole 12b. More specifically, the second housing 202 may penetrate the insertion hole 12b.

The second housing 202 may be fixed to a fixing cover 290 disposed outside the storage compartment 30 in a state of penetrating the insertion hole 12b. The fixing cover 290 may be disposed outside the inner case 12. Specifically, the housing 201 includes a coupling hook 202a formed on an upper surface of the second housing 202, and as the coupling hook 202a is hook-coupled to a corresponding coupling protrusion 292 when the second housing 202 penetrates the insertion hole 12b, the housing 201 may be fixed to the fixing cover 290.

When the housing 201 is fixed to the fixing cover 290, the purifier assembly 200 may be fixed to the upper surface 12a of the inner case 12. When the purifier assembly 200 is fixed to the upper surface 12a of the inner case 12, the first housing 203 may be disposed in the storage compartment 30, and a portion of the second housing 202 may be disposed outside the storage compartment 30 by penetrating the inner case 12. When the purifier assembly 200 is fixed to the upper surface 12a of the inner case 12, a flow path part 202b and an electronic component accommodating part 202c of the second housing 202 may be inserted into an insertion space 291 of the fixing cover 290 outside the storage compartment 30.

That is, when the purifier assembly 200 is fixed to the upper surface 12a of the inner case 12, as the housing 201 penetrate the inner case 12, a portion of the housing 201 may be disposed inside the storage compartment 30, and the remaining portion of the housing 201 may be disposed outside the storage compartment 30. Therefore, the extent to which the purifier assembly 200 occupies the storage compartment 30 may be minimized, and a flow of cold air in the storage compartment 30 may be minimized from being obstructed by the purifier assembly 200. In addition, aesthetics may be improved by minimizing a volume of the purifier assembly 200 that may be seen by the user when the doors 20 and 21 are opened.

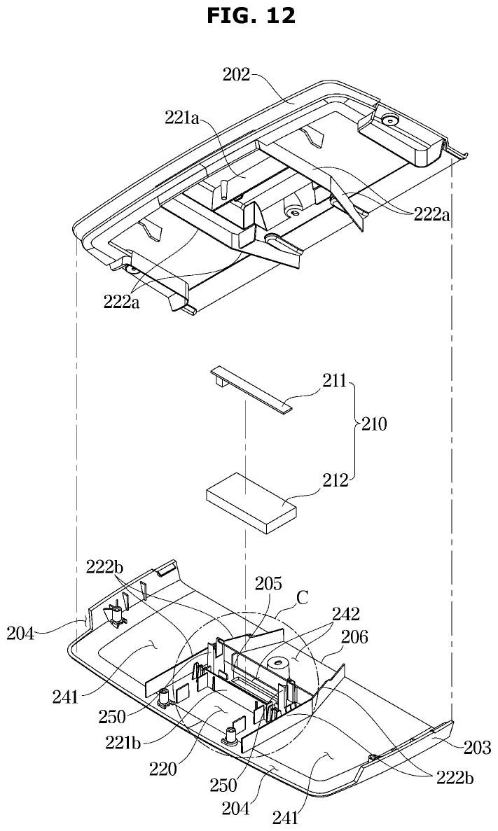

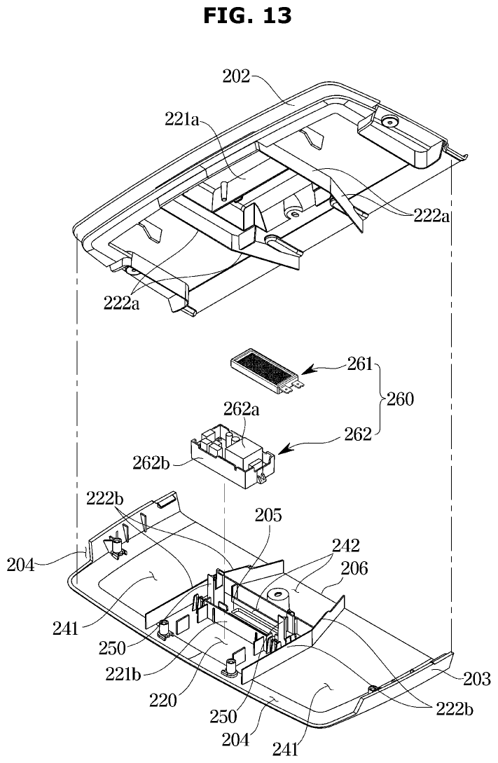

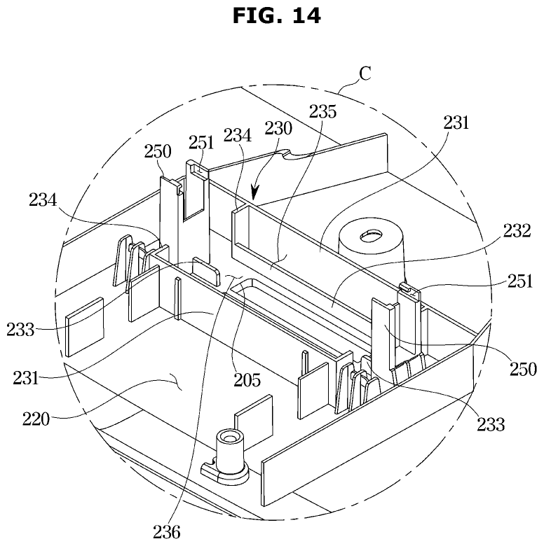

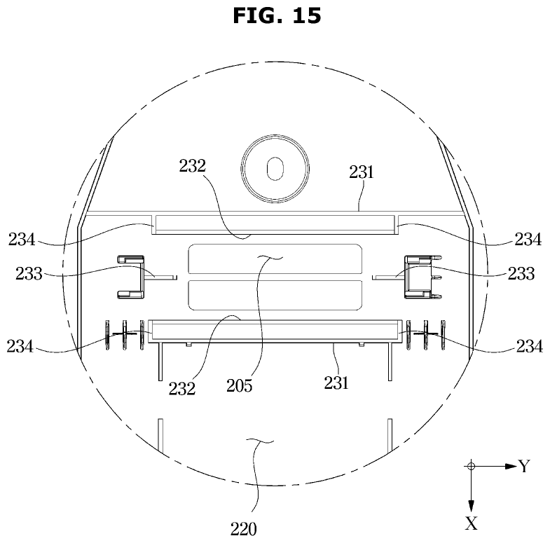

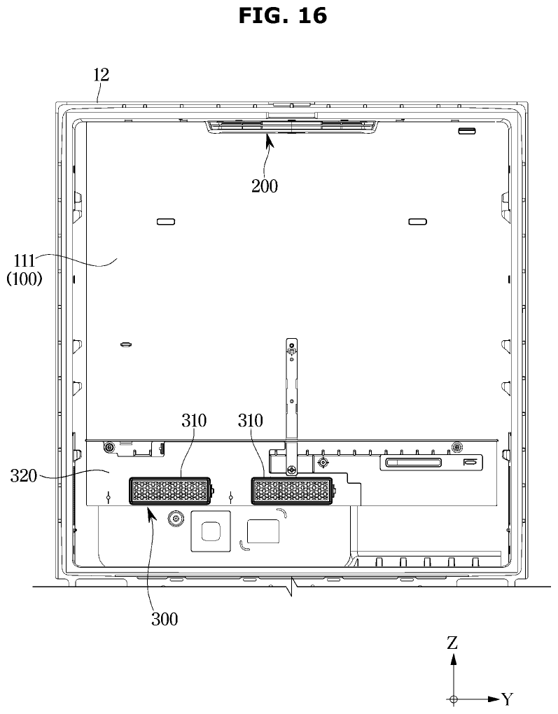

FIG. 5 is an enlarged view of part B shown in FIG. 4. FIG. 12 is an exploded perspective view illustrating a state in which a photocatalytic device is mounted in the purifier assembly illustrated in FIG. 6. FIG. 13 is an exploded perspective view showing a state in which an ion sterilizer is mounted in the purifier assembly illustrated in FIG. 6. FIG. 14 is an enlarged view of part C shown in FIG. 12. FIG. 15 is a top view of the C part. FIG. 16 is a view illustrating a state in which a deodorization filter is disposed in the first storage compartment illustrated in FIG. 3.

Hereinafter, an internal structure of the purifier assembly will be described in detail with reference to FIGS. 5 and 12 to 16.

The purifier assembly 200 may include a first flow path 242 and a second flow path 241 through which cold air introduced into the inlet 206 flows and which are partitioned from each other.

Specifically, the housing 201 may include a first inner wall 222b provided on the first housing 203 and a second inner wall 222a provided on the second housing 202 to correspond to the first inner wall 222b, and when the first housing 203 and the second housing 202 are coupled to each other, the first inner wall 222b and the second inner wall 222a may partition an inner space of the housing 201 into the first flow path 242 and the second flow path 241. The first flow path 242 may connect the inlet 206 and the first outlet 205, and the second flow path 241 may connect the inlet 206 and the second outlet 204. The second flow path 241 may be configured as a plurality of the second flow paths 241.

Because the air flowing in the circulation flow path 130 through the cold air discharge port 120a is introduced into the first flow path 242 and the second flow path 241 inside the purifier assembly 200, it may be seen that the first flow path 242 and the second flow path 241 are part of the circulation flow path 130, and the circulation flow path 130 is branched into the first flow path 242 and the second flow path 241 in the housing 201. It may be seen that the purifier assembly 200 is disposed on the circulation flow path 130.

The air purifiers 210 and 260 may be disposed on the first flow path 242 to deodorize and/or sterilize cold air flowing through the first flow path 242. Specifically, the second housing 202 may include a flow path part 202b forming the first flow path 242 having a height greater than the other portion of the first flow path 242 so that the air purifiers 210 and 260 may be disposed therein, and the air purifiers 210 and 260 may be disposed on the first flow path 242 formed between the flow path part 202b of the second housing 202 and the first housing 203. The flow path part 202b may be formed by protruding a portion of the upper surface of the second housing 202 upward.

That is, the purifier assembly 200 may circulate a part of cold air into the storage compartment 30 through the second flow path 241 by using the flow of the cold air discharged by the duct 100, and may perform deodorization and/or sterilization of the remaining part by the air purifiers 210 and 260 through the first flow path 242.

A flow rate of cold air in the second flow path 241 that does not pass through the purifier assembly 200 may be greater than a flow rate of cold air in the first flow path 242 so that cold air circulates inside the storage compartment 30 smoothly. The flow rate of cold air discharged from the second flow path 241 to the storage compartment 30 may be greater than the flow rate of cold air discharged from the first flow path 242 to the storage compartment 30.

As such, because the purifier assembly 200 may perform air purification using a flow of air generated by the duct 100 and discharged to the cold air discharge port 120a even without a separate air circulation device that is disposed inside the storage compartment 30, miniaturization of the purifier assembly 200 may be achieved.

The purifier assembly 200 may include the air purifiers 210 and 260 provided to deodorize and/or sterilize cold air introduced into the housing 201. The air purifiers 210 and 260 may be the photocatalytic devices 210 or the ion sterilization devices 260. That is, the user may determine one of the photocatalytic device 210 and the ion sterilization device 260 as the air purifiers 210 and 260 mounted in the housing 201 as needed, and interchangeability is also possible.

The photocatalytic device 210 may include a UV light source 211 emitting ultraviolet rays and a photocatalytic filter 212 reacting with light from the UV light source 211 to deodorize and/or sterilize passing air.

The ion sterilization device 260 may include a discharge electrode 261 deodorizing and/or sterilizing air by generating ions in the air, and a printed circuit board 262a controlling the discharge electrode 261. The ion sterilization device 260 may include a substrate cover 262b covering the printed circuit board 262a, and may include a control unit 262 in which the printed circuit board 262a and the substrate cover 262b are combined. However, the present disclosure is not limited thereto, and the substrate cover 262b may be omitted. A plurality of electronic components may be mounted on the printed circuit board 262a.

The purifier assembly 200 may include a supporter 250 disposed on the first flow path 242 and protruding upward from the first housing 203 to support the UV light source 211. The supporter 250 may be configured as a pair of the supporters 250. The supporters 250 may be disposed between the flow path part 202b and the first housing 203 and covered by the flow path part 202b. The supporter 250 may include a support groove 251 provided such that the UV light source 211 is inserted and supported.

The first housing 203 may include a mounting portion 230 in which the photocatalytic filter 212 or the discharge electrode 261 is mounted and having the first outlet 205 formed on a bottom surface thereof, and the pair of supporters 250 may be disposed at the left and right sides of the mounting portion 230, respectively. The mounting portion 230 may be disposed on the first flow path 242.

When the air purifier mounted in the housing 201 is the photocatalytic device 210, the UV light source 211 may be mounted on the supporters 250 and the photocatalytic filter 212 may be mounted on the mounting portion 230. The UV light source 211 may be disposed above the photocatalytic filter 212 as it is mounted on the supporters 250 and may irradiate light toward the photocatalytic filter 212. Because the first outlet 205 is disposed below the photocatalytic filter 212, air flowing through the first flow path 242 may be deodorized and/or sterilized while passing through the photocatalytic filter 212 and discharged to the storage compartment 30 through the first outlet 205.

The purifier assembly 200 may include an electronic component room 220 in which the control unit 262 is disposed. The electronic component room 220 may be formed by the electronic component accommodating part 202c formed by protruding a portion of the upper surface of the second housing 202 upward to secure a space in which the control unit 262 may be accommodated. Specifically, the electronic component room 220 may correspond to a space formed between the electronic component accommodating part 202c and the first housing 203.

The electronic component room 220 may be partitioned from the first flow path 242 and the second flow path 241. Specifically, the housing 201 includes a first partition wall 221a provided in the first housing 203 and a second partition wall 221b provided in the second housing 202 to correspond to the first partition wall 221a, and when the first housing 203 and the second housing 202 are coupled to each other, the first partition wall 221a and the second partition wall 221b may partition the electronic component room 220 and the first flow path 242, and the first partition wall 221a and the second partition wall 221b may partition the electronic component room 220 and the second flow path 241. The electronic component room 220 may be disposed in front of the first flow path 242.

When the air purifier mounted in the housing 201 is the ion sterilization device 260, the control unit 262 may be accommodated in the electronic component room 220, and the discharge electrode 261 may be mounted on the mounting portion 230. Because the first outlet 205 is disposed below the discharge electrode 261, air flowing through the first flow path 242 may be deodorized and/or sterilized while passing through the discharge electrode 261 and discharged to the storage compartment 30 through the first outlet 205.

The mounting portion 230 may include a plurality of support walls 231, 232, 233, and 234. Specifically, the mounting portion 230 may include front and rear support walls 231 for the filter, lateral support walls 234 for the filter, front and rear support walls 232 for the electrode, and lateral support walls 233 for the electrode.

The front and rear support walls 231 for the filter may be configured as a pair of the front and rear support walls 231 for the filter that are parallel in left and right directions. The front and rear support walls 232 for the electrode may be configured as a pair of the front and rear support walls 232 for the electrode that are parallel in the left and right directions. The pair of front and rear support walls 231 for the filter may extend in the left and right directions. The pair of front and rear support walls 232 for the electrode may extend in the left and right directions. The front and rear support walls 231 for the filter disposed relatively at the front among the pair of front and rear support walls 231 for the filter may correspond to the first partition wall 221a.

Heights of the front and rear support walls 231 for the filter may be higher than heights of the front and rear support walls 232 for the electrode. The front and rear support walls 231 for the filter and the front and rear support walls 232 for the electrode may be parallel in the left and right directions. The pair of front and rear support walls 232 for the electrode may be disposed between the pair of front and rear support walls 231 for the filter. The lateral support walls 234 for the filter may connect one ends of the front and rear support walls 231 for the filter and one ends of the front and rear support walls 232 for the electrode adjacent to each other. The lateral support walls 233 for the electrode may be disposed between the pair of front and rear support walls 232 for the electrode. Heights of the lateral support walls 233 for the electrode and the heights of the front and rear support walls 232 for the electrode may be substantially the same.

When the photocatalytic filter 212 is mounted in the housing 201, front and rear surfaces of the photocatalytic filter 212 may be supported by the front and rear support walls 231 for the filter, left and right surfaces of the photocatalytic filter 212 may be supported by the lateral support walls 234 for the filter, and a lower surface of the photocatalytic filter 212 may be supported by the front and rear support walls 232 for the electrode and the lateral support walls 233 for the electrode. A space located above the front and rear support walls 232 for the electrode and the lateral support walls 233 for the electrode and in which the photocatalytic filter 212 is mounted so as to be supported by the front and rear support walls 231 for the filter and the lateral support walls 234 for the filter may be referred to as a filter mounting space 235.

When the discharge electrode 261 is mounted in the housing 201, front and rear surfaces of the discharge electrode 261 may be supported by the front and rear support walls 232 for the electrode, left and right surfaces of the discharge electrode 261 may be supported by the lateral support walls 233 for the electrode, and a lower surface of the discharge electrode 261 may be supported by the bottom surface of the mounting portion 230 on which the first outlet 205 is formed. A space in which the discharge electrode 261 is mounted so as to be supported by the front and rear support walls 231 for the electrode and the lateral support walls 233 for the electrode may be referred to as an electrode mounting space 236.

As such, because any one of the photocatalytic device 210 and the ion sterilization device 260 may be mounted without any change in structure, the user may easily change a type of air purifier and a manufacturing cost and production cost of a product may be reduced.

The refrigerator 1 may further include a deodorization filter assembly 300 as illustrated in FIG. 16. The deodorization filter assembly 300 may include a deodorization filter 310 containing activated carbon and a mounting plate 320 to which the deodorization filter 310 is fixed and coupled to the duct 100. The deodorization filter 310 may be configured as a plurality of the deodorization filters 310.

The deodorization filter assembly 300 may be disposed on the rear surface of the storage compartment 30 as the mounting plate 320 is coupled to a lower end of the duct 100. The deodorization filter assembly 300 may deodorize the air inside the storage compartment 30. The deodorization filter assembly 300 may be detachably mounted in the duct 100.

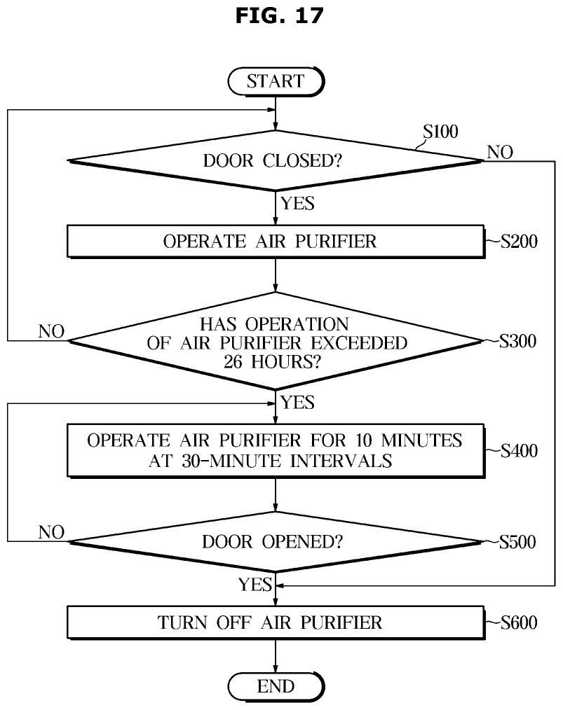

FIG. 17 is a flowchart illustrating an operation sequence of the purifier assembly illustrated in FIG. 3. Hereinafter, an operation of the purifier assembly will be described in detail with reference to FIG. 17.

A sensor of the refrigerator 1 may recognize whether the doors 20 and 21 are opened or closed (S100). When the doors 20 and 21 of the refrigerator 1 are closed, the air purifiers 210 and 260 may operate (S200). When the doors 20 and 21 of the refrigerator 1 are not closed, the air purifiers 210 and 260 may be turned off (S600). That is, when the doors 20 and 21 of the refrigerator 1 are opened, the air purifiers 210 and 260 may be turned off.

When the operation of the air purifiers 210 and 260 exceeds 26 hours (S300), mode conversion may be executed after that so that the air purifiers 210 and 260 operate for 10 minutes at 30-minute intervals (S400).

When the operation of the air purifiers 210 and 260 does not exceed 26 hours, the refrigerator 1 may recognize whether the doors 20 and 21 are opened or closed again (S100), and when the doors 20 and 21 are not opened, the air purifiers 210 and 260 may continue to operate (S200). At this time, when the doors 20 and 21 of the refrigerator 1 are opened, the air purifiers 210 and 260 may be turned off (S600).

When the doors 20 and 21 are opened in a state where the air purifiers 210 and 260 operate for 10 minutes at 30-minute intervals (S500), the air purifiers 210 and 260 may be turned off (S600). When the doors 20 and 21 are not opened in the state where the air purifiers 210 and 260 operate for 10 minutes at 30-minute intervals (S500), the air purifiers 210 and 260 may continue to operate for 10 minutes at 30-minute intervals (S400).

When the air purifiers 210 and 260 are turned off, the operation sequence of FIG. 17 may be repeated again from the operation of recognizing whether the doors are open or closed.

When power is supplied to the refrigerator 1 in a state where power is not supplied thereto, the air purifiers 210 and 260 may operate for 20 minutes. After that, the operation sequence of FIG. 17 may be repeated.

According to one or more embodiments, an extent to which a purifier assembly occupies a storage compartment may be minimized, and a flow of cold air in the storage compartment can be minimized from being obstructed by the purifier assembly.

According to one or more embodiments, an purifier assembly may perform air purification using a flow of air generated by a duct and discharged to a cold air discharge port even without a separate air circulation device.

According to one or more embodiments, aesthetics may be improved by minimizing an area of an exterior of the purifier assembly that can be seen by a user when a door is opened.

According to one or more embodiments, a user may easily change a type of air purifier and a manufacturing cost and production cost of a product may be reduced.

Although specific embodiments have been shown and described, it should be understood by those of skilled in the art that the disclosure is not limited to the above-described embodiments, and various changes and modifications may be made without departing from the technical idea of the disclosure described in the following claims.

What is claimed is:

1. A refrigerator comprising:
an inner case defining a storage compartment;
a cold air outlet provided on one side of the storage compartment and configured to discharge cold air; and
a purifier assembly provided at an upper surface of the storage compartment and configured to purify a portion of the cold air discharged from the cold air outlet,
wherein the purifier assembly comprises:
an air purifier configured to deodorize or sterilize the cold air; and
a housing accommodating the air purifier and an inlet facing the cold air outlet,
wherein the inlet is configured to receive the cold air discharged from the cold air outlet,
wherein the purifier assembly penetrates the inner case, and
wherein a portion of the housing is provided inside the storage compartment and a remaining portion of the housing is provided outside the storage compartment.

2. The refrigerator of claim 1, wherein the purifier assembly further comprises a first flow path and a second flow path in which the cold air received by the inlet flows and which are partitioned from each other, and
wherein the air purifier is provided on the first flow path and is further configured to deodorize or sterilize the cold air flowing through the first flow path.

3. The refrigerator of claim 2, wherein the air purifier further comprises at least one of (i) a photocatalytic device comprising a ultraviolet (UV) light source and a photocatalytic filter configured to react with light from the UV light source, or (ii) an ion sterilization device comprising a discharge electrode and a printed circuit board configured to control the discharge electrode.

4. The refrigerator of claim 3, wherein the air purifier comprises the photocatalytic device, and
wherein the purifier assembly further comprises a supporter provided on the first flow path and configured to support the UV light source to be disposed above the photocatalytic filter.

5. The refrigerator of claim 3, wherein the purifier assembly further comprises an electronic component room partitioned from the first flow path and the second flow path,
wherein the air purifier comprises the ion sterilization device,
wherein the discharge electrode is provided on the first flow path, and
wherein the printed circuit board is provided in the electronic component room.

6. The refrigerator of claim 3, wherein the housing further comprises a mounting portion on which the photocatalytic filter or the discharge electrode is mounted, and
wherein the purifier assembly further comprises an outlet provided on a bottom surface of the mounting portion and through which the cold air in the first flow path is discharged to the storage compartment.

7. The refrigerator of claim 1, wherein the inner case comprises an insertion hole, and
wherein the housing penetrates the insertion hole.

8. The refrigerator of claim 7, further comprising a fixing cover provided outside the inner case and to which the housing penetrating the insertion hole is fixed.

9. The refrigerator of claim 8, wherein the housing further comprises a coupling hook provided on an upper surface of the housing and configured to be coupled to the fixing cover.

10. The refrigerator of claim 1, further comprising: a duct comprising a circulation flow path configured to circulate the cold air in the storage compartment and a fan provided on the circulation flow path,
wherein the cold air outlet is provided at an upper end of one surface of the duct forming a rear surface of the storage compartment, and
wherein the inlet is configured to receive the cold air discharged from the fan.

11. The refrigerator of claim 10, wherein the inlet is provided inside the storage compartment.

12. The refrigerator of claim 11, wherein the inlet is adjacent to the cold air outlet.

13. The refrigerator of claim 2, wherein a flow rate of the cold air discharged from the second flow path to the storage compartment is greater than a flow rate of the cold air discharged from the first flow path to the storage compartment.

14. The refrigerator of claim 2, wherein a direction in which the cold air is discharged from the first flow path to the storage compartment is different from a direction in which the cold air is discharged from the second flow path to the storage compartment.

15. The refrigerator of claim 1, further comprising a deodorization filter provided on a rear surface of the storage compartment.
